(19) United States
(12) Patent Application Publication
(10) Pub. No.: US 2026/0013718 A1
(43) Pub. Date: Jan. 15, 2026

(54) OPHTHALMIC SURGERY OPERATING SYSTEM, COMPUTER PROGRAM AND METHOD FOR PROVIDING ASSESSMENT INFORMATION CONCERNING THE GUIDANCE OF A SURGICAL TOOL

(71) Applicant: Carl Zeiss Meditec AG, Jena (DE)

(72) Inventors: Martin HACKER, Jena (DE); Christoph HAUGER, Oberkochen (DE); Karlheinz REIN, Oberkochen (DE)

(21) Appl. No.: 19/137,641

(22) PCT Filed: Oct. 30, 2023
(86) PCT No.: PCT/EP2023/080286

§ 371 Date: Jun. 10, 2025

(30) Foreign Application Priority Data
Dec. 12, 2022 (DE) 10 2022 133 005.2

(51) Int. Cl. A61B 3/117

(52) U.S. Cl. CPC A61B 3/117

(57) ABSTRACT

An ophthalmic surgery operating system comprises a surgical tool to act on a region of action, which is a spatially extended region of possible actions of the surgical tool within a time window of action. A computer program includes a surgical tool program routine for providing a model of the region of action and a routine for determining the spatial position of the model of the operating site with respect to the model of the region of action from the reference measurement data. The computer program has a prognosis routine that determines, from the spatial position of the model of the operating site with respect to the spatial position of the model of the region of action, a continuously adapted model, valid for a time interval that includes the time window of action, concerning the predicted result of the surgical operation on the patient's eye.

### CROSS-REFERENCE TO RELATED APPLICATIONS

The present application is the U.S. National Stage entry of International Application No. PCT/EP2023/080286, filed Oct. 30, 2023, which claims priority to German Patent Application No. 10 2022 133 005.2, filed Dec. 12, 2022, the contents of which are incorporated by reference herein in their entirety.

### DESCRIPTION

The invention relates to an eye surgery operating system for performing a surgical operation in an operating site on a patient's eye, having a surgical tool with which it is possible to have an effect on tissue structures of the patient's eye arranged in an area of effect that is a spatially extensive region of possible effects due to the surgical tool within a time window of action, and having a computer unit which contains a program memory with a computer program, the latter comprising an operating site model program routine for providing a model of the operating site, and which is embodied to continually acquire referencing measurement data concerning the patient's eye and concerning the surgical tool. The invention also relates to a computer program for providing assessment information relating to the guidance of a surgical tool in a surgical operation on a patient's eye and to a computer-implemented method for providing assessment information relating to the guidance of a surgical tool in a surgical operation on a patient's eye.

An eye surgery operating system of the type set forth at the outset is known from DE 10 2020 102 011 A1. This eye surgery operating system contains an OCT apparatus that serves to capture the pose of a surgical tool in a model of a patient's eye, which can be displayed to a surgeon as a 3-D reconstruction of a region of the patient's eye. The display unit allows for the display of an actual and a target position for the surgical tool.

WO 2019/170669 A1 describes the creation of control data for an ophthalmological laser therapy device, which serves to create a structure that reduces the intraocular pressure in the tissue of the patient's eye and bridges the cornea.

Displaying to a surgeon a piece of position information referenced to a patient's eye and concerning a surgical incision or surgical incisions within the scope of an eye surgery operating system was disclosed in DE 10 2018 124 065 A1.

U.S. Pat. No. 10,842,573 B2 describes an eye surgery operating system containing a computer unit for creating a calculation model for assisting eye surgeons, the model allowing an estimation of the load on the retina during membrane peeling.

US 2018/000339 A1 specifies the ascertainment of the model of a patient's eye in an ophthalmological operation on account of data acquired in surgery, in order to display information concerning the model to a surgeon in the ophthalmological operation.

As regards the insertion of intraocular lenses into a patient's eye, surgical tool guiding templates that assist a surgeon with guiding surgical tools are known and aligned using markings attached to a patient's eye pre-surgery.

The problem addressed by the invention is that of providing an eye surgery operating system and specifying a computer program and a method, which increase the precision of surgical procedures on a patient's eye.

This problem is solved by an eye surgery operating system, a related computer program, and a related method for performing a surgical operation in an operating site on a patient's eye. Advantageous embodiments and developments of the invention are disclosed herein.

An eye surgery operating system according to the invention for performing a surgical operation in an operating site on a patient's eye contains a surgical tool with which it is possible to have an effect on tissue structures of the patient's eye that are arranged in an area of effect, wherein the area of effect is a spatially extensive region of possible effects due to the surgical tool within a time window of action. The eye surgery operating system has a computer unit which contains a program memory with a computer program that comprises an operating site model program routine for providing a model of the operating site. The computer unit is embodied to continually acquire referencing measurement data concerning the patient's eye and concerning the surgical tool. The computer program has a surgical tool program routine for providing a model of the area of effect. The computer program contains a routine for determining the spatial pose of the model of the operating site relative to the model of the area of effect from the referencing measurement data. The computer program comprises a prediction routine that is developed to use the spatial pose of the model of the operating site relative to the spatial pose of the model of the area of effect to determine a continually adapted model that is in regard to the predicted result of the surgical operation on the patient's eye and valid for a time interval comprising the time window of action, i.e. to determine a model that is valid for a time interval comprising the time window of action. The computer program contains a routine for continually providing assessment information relating to the guidance of the surgical tool in the surgical operation, said routine considering the model of the area of effect and the model of the operating site and the provided, continually acquired referencing measurement data and the model regarding the predicted result of the surgical operation on the patient's eye, e.g. by virtue of the assessment information being ascertained from the model of the area of effect and the model of the operating site and the provided, continually acquired referencing measurement data and the model regarding the predicted result of the surgical operation on the patient's eye.

A basic concept of the invention lies in providing the surgeon with information in respect of whether and to what extent the surgical tool application in the current pose of the surgical tool relative to the operating site would deliver the sought-after result of the surgery, with this information being provided at all times during an eye operation. In this context, an advantage of the invention can be found in the consideration of the dynamics of the present system made up of operating site and surgical tool.

A surgical tool within the meaning of the invention is e.g. a lancet, a laser, a needle, a stabilized needle, a drill, an injection needle, a plasma cutter, an endoscope having a laser for tissue ablation or tissue coagulation, an endoscopic laser probe for tissue ablation or coagulation, an implant injector, a goniotomy cutter, a trabecular meshwork trephine, a dilation catheter that penetrates the trabecular meshwork for Schlemm's canal, or else a laser system for LASIK (laser in-situ keratomileusis).

A time window of action within the meaning of the invention is a time window within which the surgical tool has an effect on tissue structures in the patient's eye. For example, a time window of action may have a length lw, to which the following applies: 16 ps≤lw≤0.4 s, by preference 1 μs≤lw≤0.3 s or 1 ms≤lw≤0.2 s or 10 ms≤lw≤0.1 s. A time window of action within the meaning of the invention is e.g. the time window within which portions of the cornea in a patient's eye are exposed to the laser light of a LASIK laser system, in order thus to correct the patient's eye. However, a time window of action may also be the time window within which a surgeon carries out a puncturing movement into the cornea of the patient's eye using a lancet or using a needle. A time window of action may also be the time window within which a surgeon injects an implant by means of an implant injector in a patient's eye. In particular, a time window of action may be the time window within which a goniotomy incision is introduced into the cornea of a patient's eye by a surgeon using a goniotomy cutter when the cutter is placed against the cornea.

A model of an object is understood by the invention to be a construct that describes only the properties of an original considered important in order to arrive at an abstracted representation of the original that is manageable or mathematically calculable or suitable for experimental investigations as a result of this simplification. In any case, a model within the meaning of the invention describes at least the geometric shape of the object. Additionally, a model of an object within the meaning of the invention may describe properties of the object from the group of local blood flows in the object, the course of tissue in the object, in particular of blood vessels, spectral absorption of light in the object, perfusion in the object, tissue types in the object, mechanical properties of the object or mechanical properties such as pressures, stresses or elasticity in sections of the object.

A model of the operating site within the meaning of the invention may be e.g. a point cloud that describes the operating site. The model of the operating site may also describe the surface shape of a cornea of the patient's eye. In particular, a model of the operating site may be a CAD model and/or a height profile of a segment of the patient's eye and/or a distance profile of the patient's eye and/or a depth profile of the patient's eye and/or a three-dimensional surface representation of a segment of the patient's eye and/or a two-dimensional surface representation of a segment of the patient's eye.

Accordingly, a model of the area of effect of the surgical tool on the patient's eye may be a point cloud or a CAD model or a three-dimensional polyline as the description of a spatially extensive zone in particular, in which the surgical tool may act on body tissue in the patient's eye and/or on media arranged in the patient's eye.

In the present case, a model that is in regard to the predicted result of the surgical operation on the patient's eye and valid for a time interval comprising the time window of action is understood to mean a model that describes the predicted result of the surgical operation meaningfully, i.e. with a sufficient accuracy, at least for a time interval comprising the time window of action. In particular, tolerances predetermined for a valid model, for example in relation to the guidance of the surgical tool, in particular during a time window of action, may be taken into account. In other words: For a valid model, the predicted result may consider predetermined possible deviations when guiding the surgical tool, especially during the time window of action.

The invention is based on the insight that the accurate pose of the area of effect of a surgical tool used by a surgeon during a surgical procedure on a patient's eye is decisive for the success of the surgical operation undertaken therewith, i.e. for whether and to what extent the result post-surgery corresponds to the expectations.

For instance, in the case of what is known as a limbal relaxing incision, during which a modification of the astigmatism is intended to be attained by way of an incision into the limbus of the patient's eye during a cataract operation, it was found that the implementation of incisions without assessment information and purely on the basis of the experience of a surgeon or by way of the use of a pen mark on the surface of the eye in the case of inexperienced surgeons often leads to unsatisfactory results.

A corresponding statement also applies to the insertion of what are known as translimbal drainage stents for treating glaucoma, which are positioned within a translimbal or transcorneal incision in the patient's eye. In this case, the position and the angle of the incision to be performed in the patient's eye decisively determine the resultant pose of the implant in the anterior chamber between the iris and the cornea.

By preference, the following applies to the length lz of the time window of action comprising the time interval and the length lw of the time window of action:

$$lz > lw + 0.1 s$$

Particularly preferably, the following applies to the length lz of the time interval comprising the time window of action:

$$lz > L ,$$

where L is the latency time for the provision of the model regarding the predicted result of the surgical operation on the patient's eye.

It is advantageous if the operating site model program routine is designed to continually adapt the model of the operating site on the basis of the continually acquired referencing measurement data. In this way, a predicted result of the operation, capable of predicting a change in the operating site during the operation, may be supplied to a surgeon in the operation.

In particular, the model regarding the predicted result of the surgical operation on the patient's eye can be a model for the pose of an implant in the patient's eye.

The routine for continually providing the assessment information may further consider the model regarding the predicted result of the surgical operation on the patient's eye.

In particular, the assessment information relating to the guidance of the surgical tool may contain assessment information or be assessment information that results from a comparison of the model for the predicted result of the surgical operation with a reference. In that case, the assessment information is a measure for an expected success of the surgery.

In particular, the assessment information may be binary information, e.g. “move on” or “pull back”, or traffic light information, “red”, “green”, etc., for example depending on whether the assessment of the predicted result of the surgery drops below or exceeds an acceptance threshold.

The eye surgery operating system may comprise a device for indicating the assessment information, which for example indicates the assessment information as an acoustic and/or an optical and/or a haptic indication signal. The reference may be a model for an optimal result of the surgery, created for the patient's eye. The model created for the patient's eye may be based on patient data acquired pre-surgery.

A further concept of the invention is that of, for an eye operation, indicating to the surgeon in an operation scenario firstly the spatial pose of the portion of effect of a surgical tool in an object region on or in a patient's eye with the patient's eye in a three-dimensional coordinate system, e.g. in the form of a point cloud, wherein the coordinate system is referenced to the patient's eye.

A further aspect of the invention is that of indicating planning information concerning an incision guidance to the surgeon in an operation scenario. This planning information may be planning information ascertained for a patient's eye pre-surgery, wherein it is advantageous if the planning information is adapted during the operation on the basis of data obtained for the patient's eye.

The data preferably comprise three-dimensional image data that contain depth information. The computer program may be designed to adapt the planning information on the basis of structures in the three-dimensional image data captured by means of an image evaluation.

For example, the three-dimensional image data may be acquired by means of a stereo camera system, by means of a confocal scanner and/or by means of an OCT system and/or by means of a Scheimpflug camera and/or by means of an ultrasonic system. In particular, the three-dimensional image data may be composed of image data that cover spatial regions of different widths and/or of different depths and/or of different spatial resolutions and/or spatial regions detected using different spectra.

In particular, the computer program for ascertaining the pose of the portion of action of the surgical tool may contain a tracking routine, which measures characteristic surgical tool features from the group of opacity, casted shadow, edge shape, surface shape and light signals by means of image recognition and/or markings on the surgical tool.

It is advantageous for the three-dimensional data to cover the entire eye socket. What should be observed here is that the pose of the eye in the eye socket represents degrees of freedom or parameters that should be taken into account during the planning. What should also be observed here is that eyes may be positioned and secured vis-à-vis the eye socket during eye surgery, for example by means of the tools in access ports, but in part also by temporarily sutured threads. The planning information may be based on patient data ascertained pre-surgery.

It is advantageous if the computer program for ascertaining the pose of the portion of action of the surgical tool in the three-dimensional coordinate system from continually acquired three-dimensional image data concerning the object region and the patient's eye and the surgical tool uses a registration routine that considers a refraction of light at interfaces in the patient's eye and/or index gradients in the patient's eye.

For referencing the spatial pose of the model of the area of action to the model of the operating site in a coordinate system, the computer program may contain a referencing routine with a tracking routine, which assesses characteristic surgical tool features from the group of opacity, casted shadow, edge or surface shape, light signals by means of image recognition and/or markings on the surgical tool.

Three-dimensional image data are preferably provided as referencing measurement data, wherein the computer program uses a registration routine that considers a refraction of light at interfaces in the patient's eye and/or index gradients in the patient's eye.

The eye surgery operating system may comprise a magnetic tracking system, wherein the referencing measurement data contain location data concerning the surgical tool that were acquired by means of the magnetic tracking system.

In particular, the model of the operating site may be a model from the group of a point cloud that describes the operating site, a surface shape of a cornea of the patient's eye, a CAD model, a height profile of a segment of the patient's eye, a distance profile of the patient's eye, a depth profile of the patient's eye, a three-dimensional surface representation of a segment of the patient's eye, a two-dimensional surface representation of a segment of the patient's eye or a model combined from the models specified above.

The model of the area of effect can also be a point cloud that describes a zone in which the surgical tool can have an effect on body tissue in the patient's eye and/or on media arranged in the patient's eye. In addition to the coordinates of the area of effect, the points of the point cloud may also describe as a matter of principle properties of the area of effect, e.g. the color of tissue, tissue annotations, calculated or measured mechanical properties such as e.g. stresses, pressure, etc.

The computer program may contain a routine that serves to continually provide displacement information relating to the guidance of the surgical tool in the surgical operation and is ascertained from the model of the area of effect and the model of the operating site and the provided, continually acquired referencing measurement data.

The displacement information for the guidance of the surgical tool may be information from the group of spatial pose of the surgical tool and direction for the displacement of the surgical tool in the coordinate system of the eye surgery operating system or relative to the model of the operating site.

The eye surgery operating system may comprise a device for indicating the information from the group of spatial pose of the surgical tool and direction for the displacement of the surgical tool in the coordinate system of the eye surgery operating system or relative to the model of the operating site as an acoustic and/or an optical and/or a haptic indication signal.

A further aspect of the invention also consists of planning an operation on the basis of data with image information concerning the patient's eye acquired pre-surgery, the scope of said plan containing a definition for this image information of a preferred application pose for the surgical tool or a preferred pose range for the surgical tool. In particular, an idea of the invention is that of registering image information concerning eye structures obtained in surgery with image information regarding eye structures acquired pre-surgery in order to adapt the preferred application pose for the surgical tool or the preferred pose range for the surgical tool and to track deviations from a preferred application pose and indicate these to a surgeon.

In this case, the image information acquired pre-surgery and in surgery can be registered e.g. by way of a nonlinear coordinate transformation, by means of which the pose deviations of corresponding landmarks in the eye structures are minimized. By subjecting an application pose for a surgical tool pre-surgery to a nonlinear coordinate transformation in particular, it is possible to display the application pose for the surgical tool with a corrected pose for image information acquired in surgery.

One aspect of the invention is that of indicating to the surgeon the displacement information as a movement to be carried out using the surgical tool, e.g. an incision movement of a lancet, using a current position of the portion of action of the surgical tool as a starting point.

In particular, an idea of the invention is that a pose of a surgical tool tracked in surgery is used to this end in order to determine a hypothetical pose for the surgical tool or a hypothetical pose range for the surgical instrument that should be expected for a specific movement of the surgical tool when its actual position is used as a starting point.

The invention proposes that a hypothetical application pose for a surgical tool vis-à-vis an application pose planned pre-surgery is indicated to a surgeon as assessment information or displacement information.

Moreover, to ascertain the assessment information, the invention proposes a determination and assessment of a hypothetical result of the surgery on the basis of a hypothetical movement of the surgical tool.

For example, the limbal relaxation incision realizable in the case of a hypothetical forward motion from a current position and alignment of a surgical tool that takes the form of a scalpel may be assessed in relation to the resultant astigmatism correction, e.g. with regard to the strength and angles in the tolerance range about the target values.

Also, indicating the assessment information as information concerning the continually adapted model regarding the predicted result of the surgery on the patient's eye in the form of an assessment of an implant offset resulting from a hypothetical puncture position and puncture direction of a translimbal drainage implant introduced using a surgical tool that takes the form of an implant injector, in respect of sufficient distances from sensitive eye structures such as corneal endothelium or iris, is possible according to the invention.

In particular, it is also possible to indicate the assessment information as an assessment in respect of the refractive results of the expected offset of IOLs fixed to the sclera, on the basis of an assessment of hypothetical puncture sites for the attachment of fixing threads.

Within the scope of the invention, it is also possible to indicate the assessment information on the basis of an assessment of the expected mechanical unburdening of the retina for the hypothetical local vitreous dissection in the case of vitreotractions.

Within the scope of the invention, it is also possible to indicate the assessment information on the basis of an assessment of the expected reduction in the intraocular pressure IOP in the patient's eye on account of a hypothetical application of a surgical tool that takes the form of a needle or an endoscopic excimer laser probe or a Schlemm's canal stent or trabecular meshwork shunt injector, for a specific tool position, for instance in relation to collector vessel positions or collector vessel alignments or in relation to the position of the trabecular meshwork.

Within the scope of the invention, it is also possible to indicate the assessment information on the basis of an assessment of the expected IOP reduction on account of the strength of application of a hypothetical application of a surgical tool that takes the form of a needle or an endoscopic excimer laser probe, i.e. the expected size of holes created in the trabecular meshwork by ablation.

Finally, it is possible within the scope of the invention to create the assessment information on the basis of the current, optionally disadvantageous position of the surgical tool and the alignment thereof by virtue of a warning being issued and a warning signal being generated prior to a membrane tear in the case of a hypothetical capsulorhexis implementation on the basis of a current, optionally disadvantageous position of the surgical tool and the alignment thereof.

Moreover, it is possible to create the assessment information on the basis of a current, optionally disadvantageous position and alignment of a surgical tool that takes the form of tweezers, in order thereby to issue a warning e.g. prior to a retinal tear with bleeding in the case of a hypothetical membrane peeling movement based on a current, optionally disadvantageous position and alignment of a surgical tool that takes the form of tweezers.

For the creation of the guide information, it is moreover also an aspect of the invention to analyze data containing image information concerning a patient's eye acquired post-surgery with image information concerning the patient's eye acquired pre-surgery and/or in surgery in order to define deviations of a planned result of an operation from an actual result of an operation by virtue of registering the image information.

In this way, it is e.g. possible to better ascertain the deviation of a planned astigmatism from one that was in fact induced by an incision or the deviation of a planned pose from an actual pose of a translimbal implant and to use this information for further result predictions pre-surgery or in surgery.

For the creation of the assessment information, it is also an aspect of the invention to use pairs with pre-surgery and post-surgery image information in a machine learning algorithm in the computer unit of the eye surgery operating system for result projections.

In particular, it is an idea of the invention that the assessment information predicts the result of a movement of the surgical tool and assesses said movement on the basis of data containing information about structures of the patient's eye. For predicting the result of the movement of the surgical tool, the invention proposes that consideration be given to forces exerted on the patient's eye, in particular by the surgical tool, or to mechanical stresses caused on the patient's eye, e.g. a stress in membranes or in the retina during what is known as membrane peeling.

To this end, the eye surgery operating system may contain a device that is coupled to the computer unit and serves to measure a force exerted by the surgical tool onto the patient's eye, wherein the routine for continually providing the displacement information considers this force for the provision of the further displacement information.

For example, forces exerted by surgical tools on eye structures may be ascertained using force sensors. For example, forces in an axial direction may be ascertained using elements that are compressible in force-dependent fashion, for instance a spring in conjunction with path measuring systems detecting the compression of said spring. Lateral forces or torsion forces may for example be ascertained by means of strain gauges attached to or integrated in the surgical tool. It is also possible to design the surgical tool in such a way that it is possible to detect a force-dependent deformation that is detectable using the imaging systems of the surgical microscope, for example the pushed-out length of a resiliently mounted surgical tool part or the lateral bend of an elastic surgical tool.

For example, forces exerted on eye structures may be implemented approximately by determining local surface deformations, e.g. by determining significant changes in the surface normal, which for example are caused by the denting of the cornea of a patient's eye just before a puncture or the lifting of a retinal membrane by means of tweezers or as a consequence of a vitreotraction.

A further option for determining forces in tissues consists in the spatially-resolved determination of compressions and stresses, i.e. the force per unit area, by means of optical coherence elastography (OCE). This is a subfield of optical coherence tomography (OCT) in which biomechanical tissue properties can be determined by virtue of local sample deformations being detected in depth-resolved fashion in a manner dependent on artificially induced compressions. In addition to determining tissue properties such as elasticity, this can be used to determine spatially resolved mechanical compressions and stresses in tissues. The necessary induced compressions can be created in such varied fashion, for example by external mechanical squeezing, mechanical vibration, ultrasonic excitation or else variation in intraocular pressure, as described in Kling et al. “Optical Coherence Elastography-Based Corneal Strain Imaging During Low-Amplitude Intraocular Pressure Modulation”, https://doi.org/10.3389/fbioe.2019.00453.

In particular, an idea of the invention is that of issuing the surgeon with a warning if there is the risk of injury to structures of the patient's eye or if there is the risk of minimum distances of the portion of action of the surgical tool or of an implant from specific structures in the patient's eye not being observed, e.g. a distance of the portion of action of the surgical tool from the iris of the patient's eye or the distance of an implant in the patient's eye from the iris of said eye.

The invention proposes to indicate such a risk to the surgeon in surgery by means of a warning signal, e.g. by means of an acoustic, an optical or a haptic warning signal. Moreover, it is possible to modify, in particular automatically modify, the configuration and/or settings of the surgical tool on the basis of the displacement information provided, for example it is possible to deactivate an active cutting function, e.g. by virtue of the cutting function of a mechanical cutter or of a laser or plasma cutter being switched off or by virtue of a lancet tip being folded-in or by virtue of a focusing setting of a laser being modified in order to modify the geometric extent of the area of effect of the laser or the preselected power of a laser or plasma cutter, wherein the time window of action may also change and the model of the area of effect may change.

In particular, it is possible on the basis of the assessment information provided to configure a surgical tool that takes the form of a blade or an injector in respect of a range for forces to be applied and penetration depths on the basis of the guide information provided or to configure the provision of a mechanical stop in order thereby to restrict a penetration of the surgical tool mechanically to certain tissue layers.

It is also an idea of the invention to ensure the penetration of the surgical tool into a certain tissue layer, e.g. the penetration into the conjunctiva or sclera or in the suprachoroidal space, for instance in order to inject a substance there.

The invention also proposes to limit, on the basis of the assessment information provided, the force exertable by the surgical tool, e.g. by electromechanically releasing a mechanical lock. However, the force exertable by the surgical tool may also be limited by virtue of the surgical tool being actively withdrawn, e.g. by means of an electromechanical, pneumatic or hydraulic drive that acts on a movable lancet tip in order thereby to avoid unwanted tissue contact.

The continually acquired data concerning the object region and the patient's eye and the surgical tool may in particular contain location data concerning the surgical tool that were acquired by means of a magnetic tracking system. The eye surgery operating system may contain a warning signal generator that serves the creation of a warning signal that depends on the ascertained position of the portion of action of the surgical tool.

It is advantageous if the computer program for determining the assessment information or the displacement information for the surgical tool considers a force exerted on structures of the patient's eye. It is also advantageous if the computer program for determining the assessment information or the displacement information considers a continually measured intraocular pressure.

In particular, it is an idea of the invention to not only acquire image information about structures of the patient's eye in surgery but also measure the intraocular pressure over time.

To this end, the eye surgery operating system may contain a device that is coupled to the computer unit and serves to measure the intraocular pressure in the patient's eye, wherein the routine for continually providing the assessment information considers the measured intraocular pressure.

For example, the intraocular pressure may be measured using an intraocular pressure measuring probe or an extraocular tonometer, for instance by means of a contact glass tonometer, an air puff, a rebound or a shockwave tonometer. Moreover, it is possible to determine the intraocular pressure at least relatively by measuring a parameter that is influenced by the intraocular pressure, for instance by measuring the corneal or scleral stress by means of OCE or by measuring the scleral curvature or corneal speckle distribution changes by means of an OCT system, as described in the publication “The effect of intraocular pressure elevation and related ocular biometry changes on corneal OCT speckle distribution in porcine eyes”, https://doi.org/10.1371/journal.pone.0249213 by M Niemczyk.

It should be observed that the temporal intervals for measuring the intraocular pressure may vary between the acquisition of image information in surgery. For example, it is possible that the acquisition of image information in surgery is triggered when predetermined values for the intraocular pressure are measured or triggered equidistantly or non-equidistantly at specific times. It is also an idea of the invention to trigger the acquisition of image information at predetermined intraocular pressure (IOP) or time values.

It should also be observed that a change of eye structure poses may be projected for yet to be measured values of the intraocular pressure or yet to be measured time values. For example, the intraocular pressure or its development over time may be inferred on the basis of a mechanical eye model from a change in eye structure poses. In this case, it is possible that the mechanical eye model has parameters that are determined from geometry-pressure combinations measured pre-surgery and in surgery, e.g. parameter values from the group of eye size and eye shape for specific different intraocular pressures, in order to then use this to infer the intraocular pressure IOP for a specific geometry of the patient's eye.

It should also be observed that the occurrence of new geometric situations can be estimated and brought to the attention of the surgeon, e.g. the situation that a dangerously low intraocular pressure is obtained in the patient's eye after the expiry of a certain time interval, e.g. after the expiry of 30 s, or the situation that the patient's eye will have sunk too deep into the eye socket, and so certain structures in the patient's eye are no longer accessible to surgical tools.

The eye surgery operating system may contain a device for irrigating the patient's eye on the basis of the acquired data concerning the patient's eye.

The eye surgery operating system may contain a micro-robot having a control unit, which receives the assessment information provided or the displacement information provided from the computer unit for the purpose of controlling the micro-robot.

The computer program according to the invention for the provision of assessment information relating to the guidance of a surgical tool in a surgical operation on a patient's eye contains an operating site model program routine for providing a model of an operating site and a surgical tool program routine for providing a model of an area of effect of a surgical tool, which describes a spatially extensive region of possible effects due to the surgical tool within a time window of action. The computer program has a routine for determining the spatial pose of the model of the operating site relative to the model of the area of effect from continually acquired referencing measurement data. The computer program contains a prediction routine that is developed to use the spatial pose of the model of the operating site relative to the spatial pose of the model of the area of effect to determine a continually adapted model that is in regard to the predicted result of the surgical operation on the patient's eye and valid for a time interval comprising the time window of action. The computer program comprises a routine for continually providing the assessment information relating to the guidance of the surgical tool, said routine considering the model of the area of effect and the model of the operating site and the continually acquired referencing measurement data and the model regarding the predicted result of the surgical operation on the patient's eye.

A computer-implemented method according to the invention for providing assessment information for the guidance of a surgical tool in a surgical operation on a patient's eye contains the following steps:
providing a model of an operating site and providing a model of an area of effect of a surgical tool, which describes a spatially extensive region of possible effects due to the surgical tool within a time window of action,
determining the spatial pose of the model of the operating site relative to the model of the area of effect from continually acquired referencing measurement data,
determining a continually adapted model that is in regard to the predicted result of the surgical operation on the patient's eye and valid for a time interval comprising the time window of action, from the spatial pose of the model of the operating site relative to the spatial pose of the model of the area of effect,
providing the assessment information relating to the guidance of the surgical tool considering the model of the area of effect and the model of the operating site and the continually acquired referencing measurement data and the model regarding the predicted result of the surgical operation on the patient's eye.

In particular, the invention is suitable for the following types of surgery:
anterior vitrectomy, i.e. the removal of the anterior part of the vitreous humor in order to prevent vitreous humor losses during cataract or corneal surgery or in order to remove displaced vitreous humor in the case of diseases such as aphakia or pupil block glaucoma;
pars plana vitrectomy or trans pars plana vitrectomy, i.e. the removal of vitreous humor opacifications and membranes by way of an incision in the pars plana;
panretinal photocoagulation;
repair of a retinal detachment;
application of a scleral buckle for repairing a retinal detachment, in order to press the sclera inwards or buckle it, as a rule by suturing a piece of conserved sclera or a piece of silicone rubber onto the surface;
laser photocoagulation or photocoagulation therapy for closing a retinal tear;
pneumatic retinopexy;
retinal cryopexy or cryotherapy in order to create a chorioretinal scar and destroy retinal or choroidal tissue;
repair of the macular hole;
partial lamellar sclerovectomy;
partial lamellar sclerocyclochoroidectomy;
partial lamellar sclerochoroidectomy;
posterior sclerotomy, i.e. the introduction of an opening into the vitreous humor through the sclera, e.g. for a retinal detachment or the removal of a foreign body;
radial optic neurotomy;
macular translocation surgery using 360° retinotomy or using the scleral imbrication technique;
refractive surgery and corneal surgery;
penetrating keratoplasty;
keratoprosthetics;
phototherapeutic keratectomy;
pterygium removal;
corneal tattooing;
osteo-odonto-keratoprosthesis;
operations for modifying the eye color by way of an iris implant, what is known as BrightOcular, or by ablating the uppermost pigment layer of the eye, what is known as the stroma procedure;
cataract surgery;
glaucoma surgery;
eye muscle surgery;
oculoplastic surgery;
operations on the lacrimal apparatus.

Below, the invention will be explained in detail on the basis of exemplary embodiments depicted schematically in the drawing,

in which:

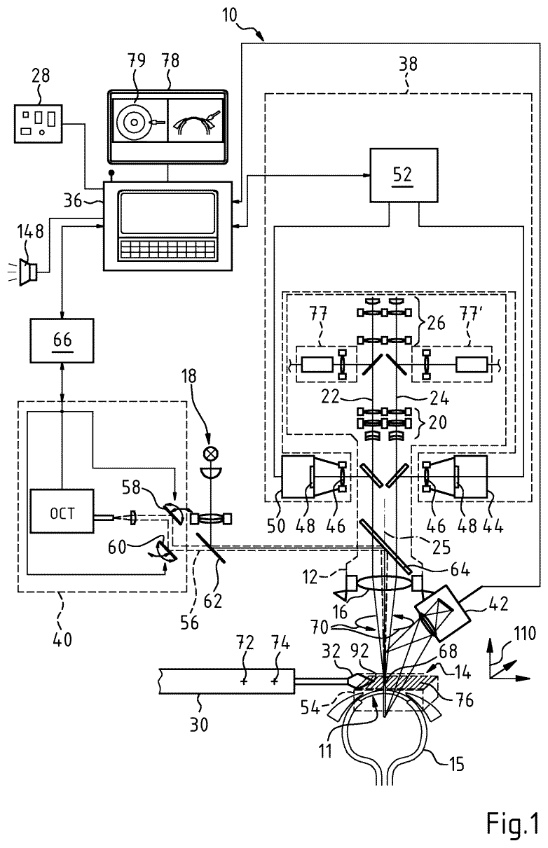

FIG. 1 shows an eye surgery operating system with a device for visualizing an operating region, with a display and with a surgical tool for surgical operation;

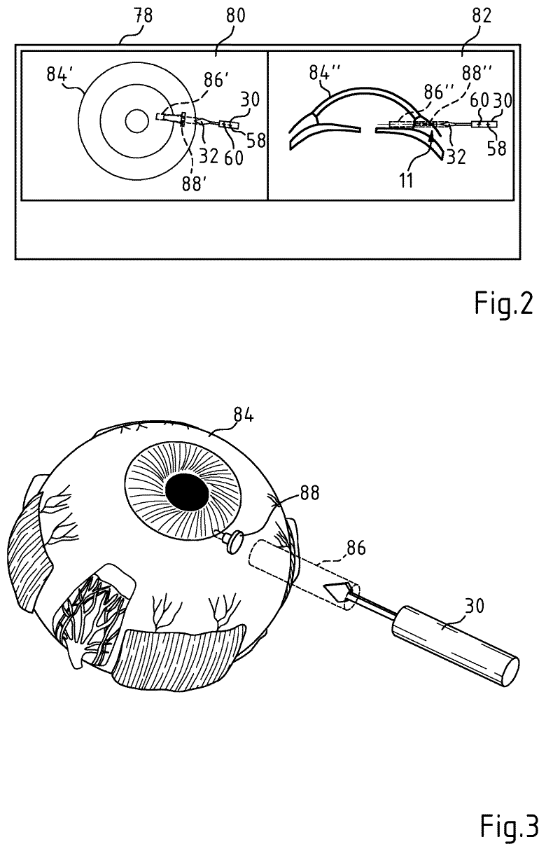

FIG. 2 shows the display of the eye surgery operating system with a digital model of the operating site on the patient's eye, a digital model of the area of effect of the surgical tool and a digital model regarding the predicted result of the surgical operation;

FIG. 3 shows a three-dimensional digital model of the operating site with a three-dimensional digital model of the area of effect of the surgical tool and a three-dimensional digital model regarding the predicted result of the operation in the form of a drainage implant arranged in a desired pose in the operating site;

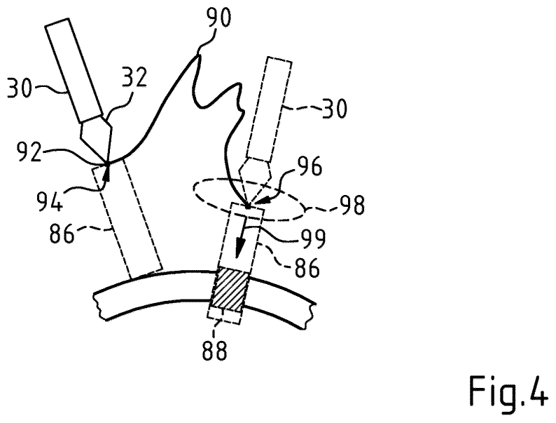

FIG. 4 shows a possible movement path along which a surgeon moves the tip of a surgical tool in the form of a lancet during a surgical operation from an initial position to an intervention position;

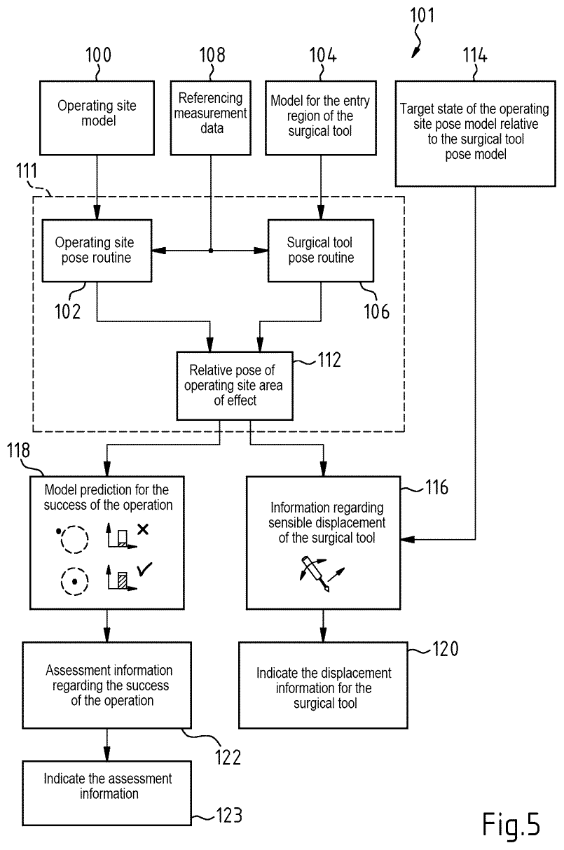

FIG. 5 shows a flowchart with program routines of a computer program loaded into a program memory of a computer unit in the eye surgery operating system;

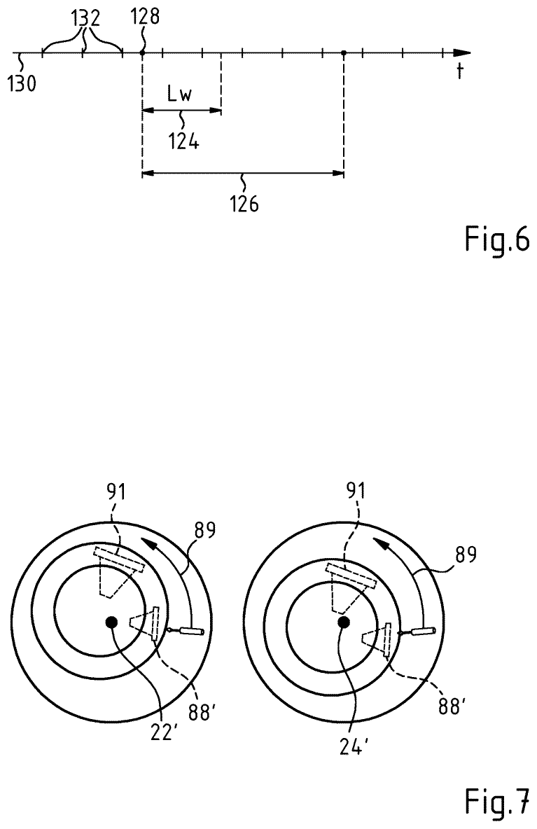

FIG. 6 shows a time window of action in a time interval to a time at which a continually adapted model regarding the predicted result of the surgical operation on the patient's eye is valid;

FIG. 7 shows an observation image for a surgeon gazing into the binocular tube;

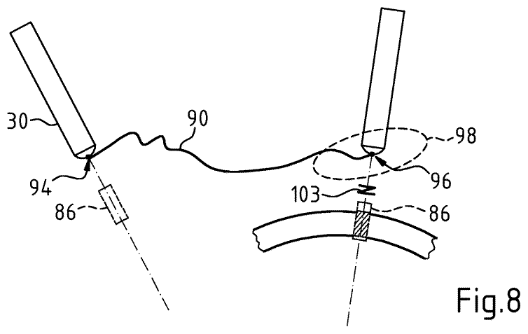

FIG. 8 shows a possible movement path along which a surgeon moves a surgical tool in the form of a laser during a surgical operation from an initial position to an intervention position;

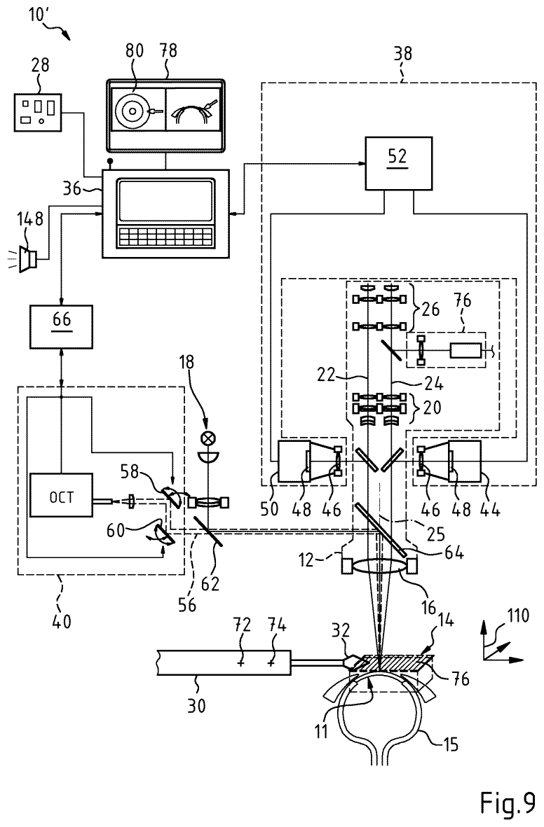

FIG. 9 shows a further eye surgery operating system with a device for visualizing an operating region, with a display and with a surgical tool for surgical operation;

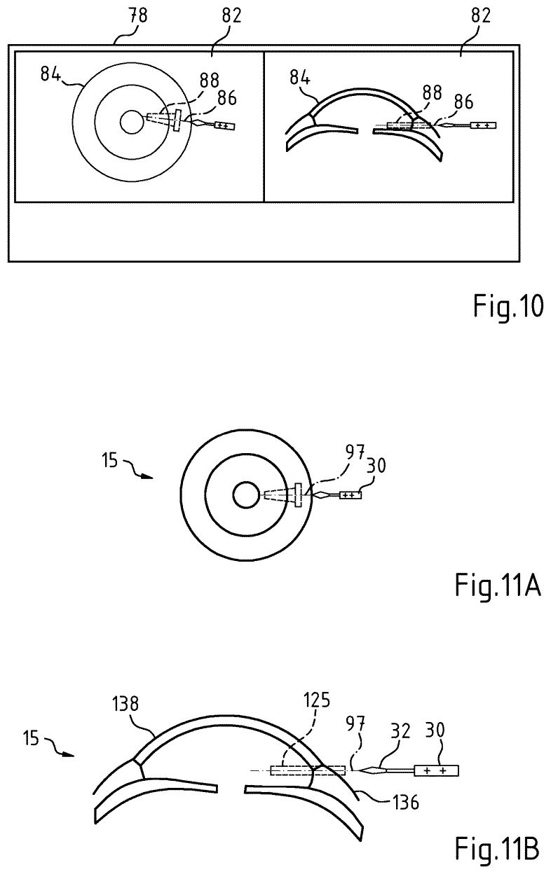

FIG. 10 shows the display of the eye surgery operating system;

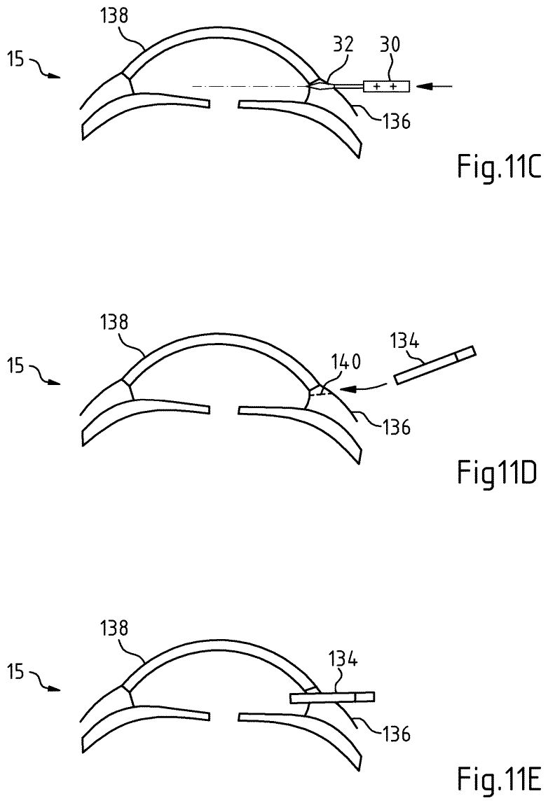

FIG. 11A to FIG. 11E show an illustration of a patient's eye in different stages of an ophthalmological operation;

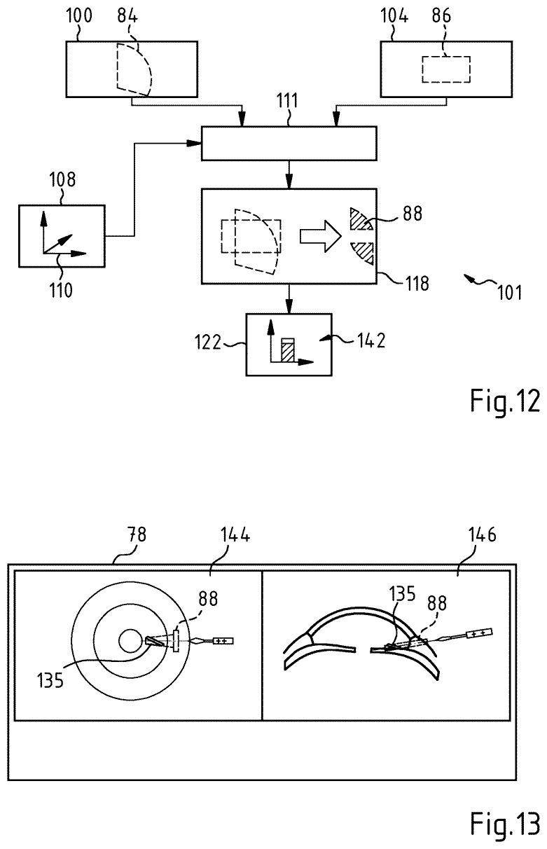

FIG. 12 shows a flowchart with program routines of a computer program loaded into a program memory of a computer unit in the further eye surgery operating system;

FIG. 13 shows the display of the further eye surgery operating system with a digital model of the operating site, a digital model of the area of effect of the surgical tool and a digital model regarding the result of the surgical operation;

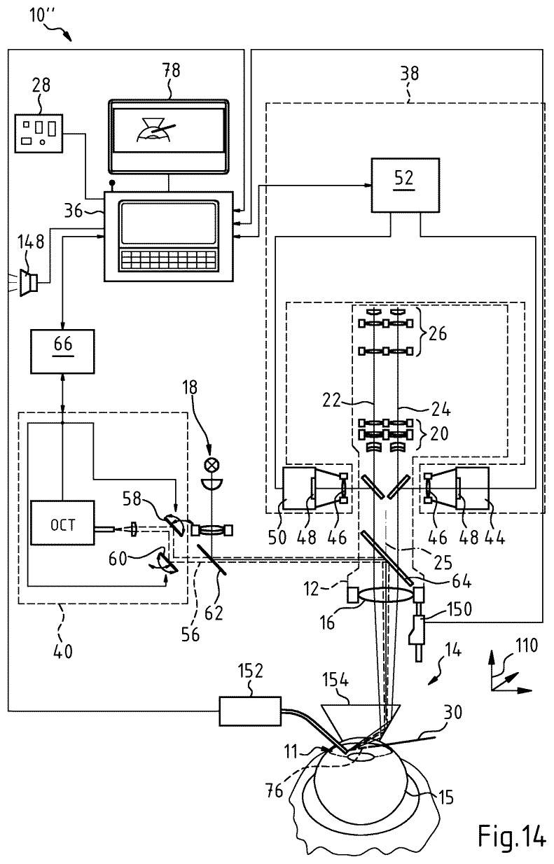

FIG. 14 shows a further eye surgery operating system with a device for visualizing an operating region, with a display and with a surgical tool for surgical operation;

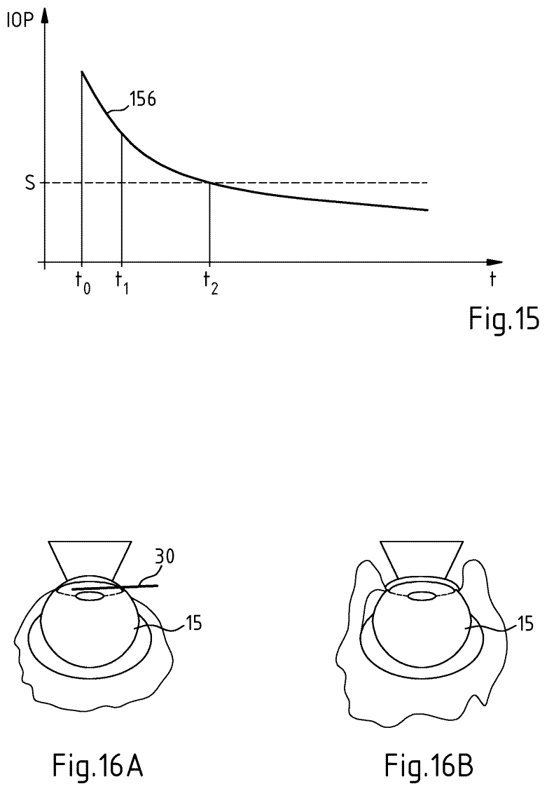

FIG. 15 shows a curve describing the change in the intraocular pressure IOP during an ophthalmological operation over time t;

FIG. 16A and FIG. 16B show an illustration of a patient's eye in different stages of an ophthalmological operation;

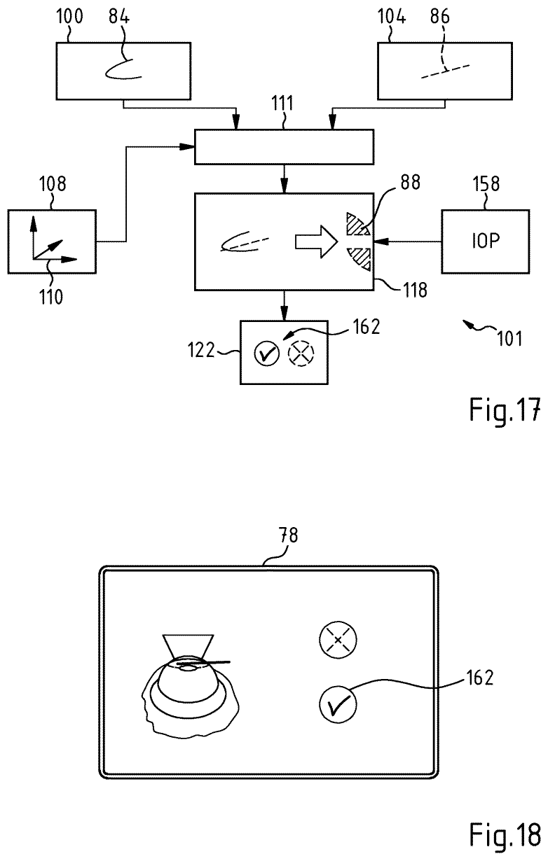

FIG. 17 shows a flowchart with program routines of a computer program loaded into a program memory of a computer unit in the further eye surgery operating system;

FIG. 18 shows the display of the further eye surgery operating system with a digital model of the operating site, a digital model of the area of effect of the surgical tool and display information for the surgeon by way of an assessment display concerning the surgical operation;

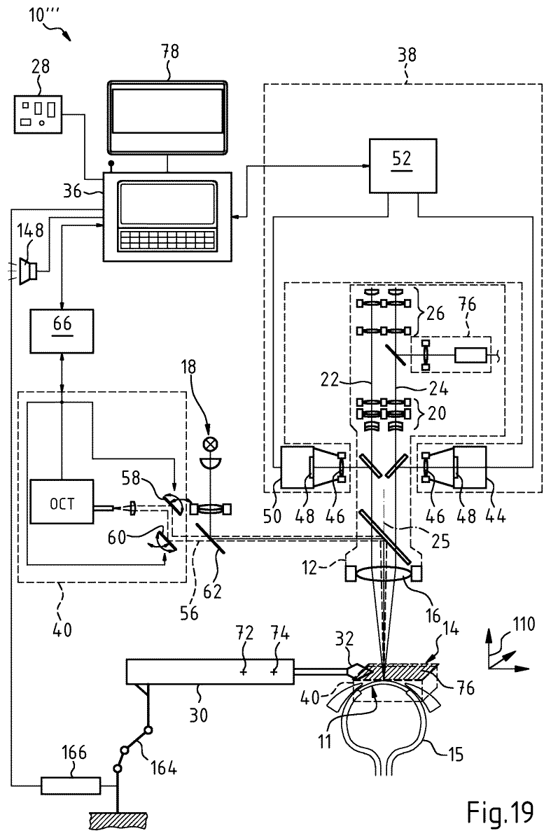

FIG. 19 shows a further eye surgery operating system with a device for visualizing an operating region, with a display and with a surgical tool for surgical operation;

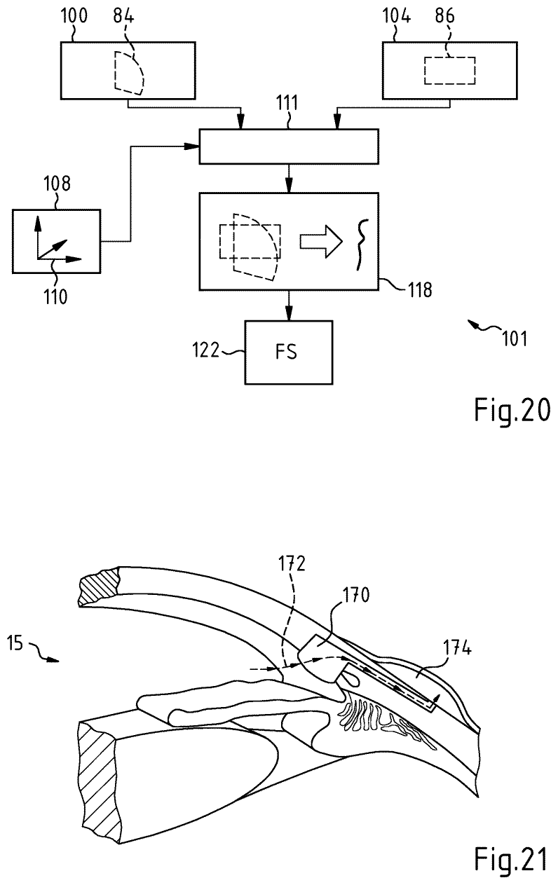

FIG. 20 shows a flowchart with program routines of a computer program loaded into a program memory of a computer unit in the further eye surgery operating system; and

FIG. 21 shows a representation of a segment of a patient's eye following a trabeculectomy.

As a device for visualizing the object region 14, the eye surgery operating system 10 shown in FIG. 1 contains a surgical microscope 12, which serves for the stereoscopic observation of an object region 14 on a patient's eye 15 with an operating site 11. The surgical microscope 12 has an imaging optics unit having a main microscope objective system 16 and accommodated in a main body. In the eye surgery operating system 10, there is an illumination device 18 that allows the illumination of the object region 14 by way of an illumination beam path passing through the main microscope objective system 16. The surgical microscope 12 comprises an afocal magnification system 20, through which a first partial stereoscopic observation beam path 22 and a second partial stereoscopic observation beam path 24 are guided. The surgical microscope 12 has a binocular tube 26 that is connected to an interface of the main body and comprises a first eyepiece and a second eyepiece for a left and a right eye of a surgeon. The main microscope objective system 16 in the surgical microscope 12 is traversed by the first partial stereoscopic observation beam path 22 and the second partial stereoscopic observation beam path 24.

The eye surgery operating system 10 includes an operating unit 28 for equipment settings and a surgical tool 30 that is designed as a lancet and has a portion of action 32 in the form of a scalpel. It should be observed that the surgical tool 30 may also take the form of a scalpel or a plasma cutter or even a laser.

In the eye surgery operating system 10, there is a computer unit 36 that is connected to an apparatus 38 for the provision of stereoscopic images with first spatial image data of the object region 14, to an OCT apparatus 40 and to a Scheimpflug camera 42.

The apparatus 38 for the provision of stereoscopic images with first spatial image data of the object region 14 comprises a first image capturing device 44 having an objective lens system 46 and having an image sensor 48 and serves to acquire data with image information from the first partial stereoscopic observation beam path 22 in the surgical microscope 12. In the apparatus 38, there is a second image capturing device 50, by means of which corresponding image information may be acquired from the second partial stereoscopic observation beam path 24 in the surgical microscope 12. To this end, the second image capturing device 50 also comprises an objective lens system 46 and an image sensor 48. In the apparatus 38, there is an image calculating stage 52 which converts data with image information from the first image capturing device 44 and from the second image capturing device 50 into spatial image data.

The OCT apparatus 40 is designed to scan an object region volume 54 on the patient's eye 15 by way of an A-scan, a B-scan and a C-scan. To scan the object region volume 54, an OCT scanning beam 56 which has short coherent light and can be moved over the object region volume 54 is created by means of the OCT apparatus 40. The OCT scanning beam 56 serves for the acquisition of data with spatial image information in the form of image data for slice recordings of the object region volume 54, as described on e.g. pages 45 to 82 of chapter 3 in A. Ehnes, “Entwicklung eines Schichtsegmentierungsalgorithmus zur automatischen Analyse von individuellen Netzhautschichten in optischen Kohärenztomographie—B Scans”, Dissertation, University of Giessen (2013).

The OCT apparatus 40 has adjustable scanning mirrors 58, 60 serving to move the OCT scanning beam 56. In the eye surgery operating system 10, the OCT scanning beam 56 is guided over beam splitters 62 and 64 and the main microscope objective system 16 into the object region volume 54 on the patient's eye 15. The light of the OCT scanning beam 56 scattered in the object region volume 54 returns at least in part to the OCT apparatus 40 via the same light path. Then, the light path of the scanning light is compared in the OCT apparatus 40 with a reference path. Using this, it is possible to detect the precise position of scattering centers in the object region volume 54, in particular the position of optically effective areas, with an accuracy which corresponds to the coherence length lc of the short coherent light in the OCT scanning beam 56. In the eye surgery operating system 10, there is a control device 66 for controlling the OCT scanning beam 56 provided by means of the OCT apparatus 40. The control device 66 allows for setting of the spatial pose and orientation of the object region volume 54 scanned in the object region 14 by means of the OCT scanning beam 56.

It should be observed that the OCT apparatus may also be designed as what is known as an SS-OCT apparatus, which serves to scan the object region with quasi-short coherent light.

The Scheimpflug camera 42 allows the acquisition of image data in a displaceable Scheimpflug camera plane 68. By means of a motor-driven drive, the Scheimpflug camera 42 is movable about the optical axis 25 of the main microscope objective system 16 in the direction of the arrows 70. By moving the Scheimpflug camera 42 about the optical axis 25 of the main microscope objective system 16, it is possible to record spatial image data concerning the object region 14 with image information that covers a portion of the interior of the patient's eye 15.

The surgical tool 30 has a first marking 72 and a second marking 74. In the eye surgery operating system, the first and second markings 72, 74 are resolved as geometric structures in the image information of the object region 14 captured both by means of the first image capturing device 44 and by means of the second image capturing device 50 when the portion of action 32 of the surgical tool 30 is located in the operating region 14.

The surgical tool 30 makes it possible to have an effect on tissue structures of the patient's eye 15 arranged in an area of effect 76 that is a region of possible effects of the surgical tool 54 within a time window of action that has an equipment-specific temporal extent determined by the handling of the surgical tool 30 by the surgeon and is considered to be time-independent in the present case. For example, the time window of action may have a length lz, to which the following applies: lz≤0.4 s. However, the following for example may also apply to the length of the time window of action: lz≤0.3 s or lz≤0.2 s or lz≤0.1 s.

The computer unit 36 in the eye surgery operating system 10 serves to control the apparatus 38 for the provision of stereoscopic images and the OCT apparatus 40 and also the Scheimpflug camera 42. It is connected to a device 77, 77′ for reflecting data into the partial stereoscopic observation beam paths 22, 24 of the surgical microscope 12, in order to allow the display of information and/or e.g. image data obtained pre-surgery in this partial observation beam path. The computer unit 36 has a program memory and is connected to a display 78 that serves to display a user interface 79.

FIG. 2 shows the display 78 of the eye surgery operating system 10 with a first representation 84′, 86′, 88′, displayed therewith, and a second representation 84″, 86″, 88″, displayed therewith, of a three-dimensional digital model 84 shown in FIG. 3 of the operating site, a three-dimensional digital model 86 shown in FIG. 3 of the area of effect of the surgical tool 30 in the surgical operation and a three-dimensional digital model 88 shown in FIG. 3 regarding the predicted result of the operation in the form of a drainage implant arranged in the operating site in a desired position.

A movement path 90 can be seen in FIG. 4, along which a surgeon moves the tip 92 of the portion of action 32 of the surgical tool 30 in the form of a lancet during a surgical operation from an initial pose 94 into an intervention pose 96 as an expedient initial pose for the surgical instrument 30 in an acceptance region 98 for performing a surgical procedure on the patient's eye.

The digital model 86 of the area of effect of the surgical tool, in which a puncture could be performed within a time interval with the typical length of 0.1 s to 0.25 s, exists in the initial pose 94. In this specific example, the puncture starting from the initial pose 94 would not reach the tissue, and it is not possible to predict a digital model 88 of an acceptable result of the surgery.

The intervention pose 96 is an advantageous initial position for the surgical instrument 30 in which the displaced digital model 86 of the area of effect of the surgical tool in that case exists on account of the spatial displacement, wherein in turn a puncture could be performed in said area of effect within a time interval with the typical length of 0.1 s to 0.25 s.

From the intervention pose 96, a surgeon is able by means of the surgical tool 30 to perform a surgical procedure on the patient's eye 15 in the form of a puncturing movement that corresponds to the arrow 99, in order thereby to bring about an incision aligned with the digital model 88 regarding the predicted result of the operation in the form of an incision in a desired pose in the operating site 11. Should the surgical tool 30 be arranged in the acceptance region 98, said surgical tool can be used to perform the intervention in the body tissue of the patient with a high probability of success, still within the time window of action with the length or duration lw.

Positioning of the surgical tool 30 in the acceptance region 98 may in particular be assisted by the use of displacement information that is ascertained from a digital model 86 of the area of effect and a digital model of the operating site and the provided, continually acquired referencing measurement data and a model regarding the predicted result of the surgical operation on the patient's eye.

To this end, the computer unit 36 in the eye surgery operating system 10 has a program memory with a computer program that comprises an operating site model program routine for the provision of the digital model 84 shown in FIG. 3 of the operating site 11. In the present case, the digital model 84 of the operating site is a CAD data model of a segment of the patient's eye, in which a surgical operation is intended to be performed.

In the computer program, there moreover is a surgical tool program routine that serves to provide the digital model 86 shown in FIG. 3 of the area of effect 76 of the surgical tool 30. Here, the digital model of the area of effect 76 of the surgical tool 30 is a CAD data model of the spatially extensive region of possible effects of the surgical tool 30 within the time window of action, during which a surgeon uses the surgical tool 30, on tissue structures arranged in the region.

The computer unit 36 receives first spatial image data of the object region 14 acquired by means of the apparatus 38 for the provision of stereoscopic images, second spatial image data of the object region 14 acquired by means of the OCT apparatus 40 and third spatial image data of the object region 14 acquired by means of the Scheimpflug camera 42 as referencing measurement data at a sampling rate rt which enables continual referencing in a coordinate system 110 of the eye surgery operating system 10 of the spatial pose of the provided digital model of the operating site 84 and of the provided digital model of the area of effect 86 in the surgical operation.

FIG. 5 shows a flowchart 101 with program routines of the computer program loaded into the program memory of the computer unit 36 in the eye surgery operating system 10.

The digital model 84 of the operating site 11 provided in the operating site model program routine 100 is supplied to an operating site pose routine 102, and the digital model 86 of the area of effect 76 of the surgical tool 30 provided in the surgical tool program routine 104 is supplied to a surgical tool pose routine 106.

In the operating site pose routine 102, the spatial pose of the digital model 84 of the operating site provided by means of the operating site model program routine 100 is determined in the coordinate system 110 of the eye surgery operating system 10 from the continually acquired referencing measurement data 108.

Accordingly, in a surgical tool pose routine 106, the spatial pose of the digital model 86 of the area of effect 76 of the surgical tool 30 is determined in the coordinate system 110 of the eye surgery operating system 10 from the provided, continually acquired referencing measurement data.

Then, the spatial actual pose of the digital model 84 of the operating site relative to the digital model 86 of the area of effect 76 of the surgical tool 30 is ascertained continually in a referencing routine 112 from the spatial pose of the digital model 84 of the operating site determined by means of the operating site pose routine 102 and from the spatial pose of the digital model 86 of the area of effect 76 of the surgical tool 30 determined by means of the surgical tool pose routine 106.

In this way, together with the referencing routine 112, the operating site pose routine 102 and the surgical tool pose routine 106 form a routine 111 for determining the spatial pose of the model 84 of the operating site relative to the model 86 of the area of effect 76 from the referencing measurement data 108. In a target state pose routine 114, a target state for the spatial pose of the digital model 86 of the area of effect 76 of the surgical tool 30 is determined in relation to the digital model 84 of the operating site.

The target state for the spatial pose of the digital model 86 of the area of effect 76 of the surgical tool 30 in relation to the digital model 84 of the operating site from the target state pose routine 114 and the relative spatial actual pose of the digital model 84 of the operating site relative to the digital model 86 of the area of effect 76 of the surgical tool 30 is then continually processed in a displacement information routine 116 to form displacement information for the surgeon moving the surgical tool 30. The displacement information is information regarding a meaningful displacement of the surgical tool 30. To this end, a distance of the target state for the spatial pose of the digital model 86 of the area of effect 76 of the surgical tool 30 in relation to the digital model 84 of the operating site from the relative spatial actual pose of the digital model 84 of the operating site to the digital model 86 of the area of effect 76 of the surgical tool 30 is determined in the displacement information routine 116, in order to ascertain from this distance displacement information containing the direction in which the surgical tool 30 must be moved in order thereby to bring about the target state for the spatial pose of the digital model 86 of the area of effect 76 of the surgical tool 30.

A continually adapted model 88 regarding the predicted result of the surgical operation on the patient's eye 15 is determined in a prediction routine 118 from the spatial pose of the digital model 84 of the operating site relative to the spatial pose of the digital model 86 of the area of effect 76 of the surgical tool 30 as determined in the routine 111.

The surgical tool 30 renders it possible to have an effect on tissue structures arranged in an area of effect 76 on the patient's eye 15, wherein the area of effect is a spatially extensive region of possible effects due to the surgical tool 30 within a time window of action 124, which can be seen in FIG. 6.

FIG. 6 shows the time window of action 124 and the time interval 126 comprising the latter at a time 128 at which the continually adapted model 88 regarding the predicted result of the surgical operation on the patient's eye 15 is valid, wherein the time window of action 124 and the time interval 126 move along the time axis 130 with an increment corresponding to the sampling rate rt, at which the referencing measurement data are provided to the computer unit 36.

The model 88 determined in the prediction routine 118 regarding the predicted result of the surgical operation on the patient's eye 15 is valid during a time interval 126 in which the time window of action 124 is located. By virtue of the model 88 regarding the predicted result of the surgical operation on the patient's eye 15 being adapted continually, the time window of action 124 and the time interval 126 move along the time axis 130 with an increment corresponding to the sampling rate rt.

Assessment information regarding the guidance of the surgical tool 30 in the surgical operation is determined, from the spatial pose of the digital model 84 of the operating site determined by means of the operating site pose routine 102 and from the spatial pose of the digital model 86 of the area of effect 76 of the surgical tool 30 determined by means of the surgical tool pose routine 106, in a routine 122 for the continual provision of assessment information relating to the guidance of the surgical tool 30. In the routine 122, this assessment information is determined by comparison with the criterion that the surgical tool 30 is arranged relative to the digital model 84 of the operating site in the acceptance region 98, from where the procedure on the body tissue of the patient can be undertaken with a satisfactory probability of success still within the time window of action of duration lw. In the routine 122, an assessment metric is applied to this end, the latter ascertaining assessment information containing the assessment information that the surgical procedure can now be performed by the surgical tool 30 with a great chance of success.

The displacement information in the displacement information routine 116 is supplied to a first display routine 120, which brings about the display of the displacement information on the display 78 in the eye surgery operating system 10.

The assessment information from the prediction routine is received by a second display routine 123 in order to display, for the surgeon, the assessment information on the display 78 in the eye surgery operating system 10.

The displacement information is a directional display on the display 78 that indicates to the surgeon the direction in which the surgical tool 30 must be moved such that a greatest possible success of the operation sets in.

The assessment information is an assessment on the display 78 of the expected result of the surgery.

The routine 118 continually references the spatial pose of the provided digital model 84 of the operating site 11 on account of the referencing measurement data 108 supplied at the referencing times 132 with respect to the spatial pose of the provided digital model 86 of the area of effect 76 in the surgical operation at a rate corresponding to the sampling rate rt in the coordinate system 110 of the eye surgery operating system 10.

In the prediction routine, the model 88 regarding the predicted result of the surgical operation on the patient's eye 15 is ascertained therefrom. Hence the model 88 is continually adapted on account of the supplied referencing data at a rate corresponding to the sampling rate, whereby the model 88 may change over time.

The time interval 126 within which the model 88 regarding the predicted result of the surgical operation on the patient's eye 15 is valid is presently understood to be a time interval in which the relative deviations of characteristic parameters of the model 88 in relation to the model at the start of the time interval are less than 10%.

Hence, the time interval is longer than the latency time L of the provision of the model 88, which is understood to mean the duration required by the routine 118 in order to specify the model 88 regarding the predicted result of the surgical operation on the patient's eye 15 from a supplied dataset of acquired referencing data concerning the digital model 84 of the operating site 11 and concerning the digital model 86 of the area of effect 76 in the surgical operation.

The extent of the time interval 126 in which the model 88 regarding the predicted result of the surgical operation on the patient's eye 15 is valid in accordance with the definition above may vary over time t. By contrast, the extent of the time window of action 126, as a specific parameter of the surgical tool 30, is invariant as a matter of principle.

The assessment information is displayed for a surgeon on the display 78 as information regarding the continually adapted model 88 regarding the predicted result of the surgical operation on the patient's eye 15 in the eye surgery operating system 10 in the form of a model 88 regarding the predicted result of the surgical operation on the patient's eye.

FIG. 7 shows an observation image for a surgeon gazing into the binocular tube 26 of the eye surgery operating system 10. The devices 77, 77′ for reflecting data into the partial stereoscopic observation beam paths 22, 24 of the surgical microscope 12 are used to display a respective representation 88′ of the model 88 which is dependent on the pose and orientation and in regard to the predicted result of the surgery to the surgeon in the left and right stereoscopic observation channels 22′, 24′ together with the displacement information 89 for the surgeon, the latter indicating to said surgeon the position into which the surgical tool 30 must be displaced for a model 91 for an optimal result of the surgery.

FIG. 8 shows a movement path 90 for a surgical tool 30 that takes the form of a laser and that is moved during a surgical operation from an initial pose 94 with the digital model 86 into an intervention pose 96 as an advantageous initial position for the surgical instrument 30 in an acceptance region 98.

On account of the spatial displacement there is a displaced model 86 of the area of effect of the surgical tool in the intervention pose 96. From the intervention pose 96, a surgeon performs a surgical procedure on the patient's eye 15 by means of the surgical tool 30 by transmitting a laser light pulse 103 within a characteristic time window of action of duration lw in order thereby to bring about a tissue ablation corresponding to a the digital model 88 regarding the predicted result of the operation in the form of a drainage implant arranged in a desired pose in the operating site 11. Should the surgical tool 30 be arranged in the acceptance region 98, said surgical tool can be used to perform the intervention in the body tissue of the patient with a high probability of success, still within the time window of action with the duration lw.

FIG. 9 shows a further eye surgery operating system 10′. To the extent that assemblies and elements of the further eye surgery microscopy system correspond to assemblies and elements of the eye surgery microscopy system 10 described above on the basis of FIG. 1 to FIG. 8, these are identified by the same numbers as reference signs.

As a surgical tool 30, the eye surgery operating system 10′ contains a lancet for introducing an incision into a connection region of the sclera and the cornea of the patient's eye 15, in which it is possible to arrange an implant by means of which fluid can be removed from the anterior chamber of the patient's eye in order to thereby reduce the intraocular pressure.

The computer unit 36 is connected to an apparatus 38 for the provision of stereoscopic images with first spatial image data of the object region 14 and to an OCT apparatus 40, which provide referencing measurement data to the computer unit.

In this case, too, the computer unit 36 receives the referencing data with a sampling rate rt that allows a continual referencing of the spatial pose of the provided digital model of the operating site and of the provided digital model of the area of effect in the surgical operation in a coordinate system 110 of the eye surgery operating system.

FIG. 10 shows the display 78 of the eye surgery operating system 10′ with a displayed first view 80 and with a displayed second view 82 of a digital model 84 of the operating site and of a digital model 86 of the area of effect of the surgical tool 30 in the surgical operation and of a digital model 88 regarding the predicted result of the operation. The digital model 88 regarding the predicted result of the operation in this case contains the pose of the implant relative to the incision into the patient's eye 15, which corresponds to the digital model 86 of the area of effect of the surgical tool 30 from the latter's current pose.

To this end, the image information from the apparatus 38 for the provision of stereoscopic images and the image information of the OCT apparatus 40 is combined by calculation in the computer unit 36 with image information from image data ascertained pre-surgery comprising a specified pose of the implant.

The computer unit 36 combines the stereoscopic images from the apparatus 38 by calculation with the image information from the OCT apparatus 40 using a registration method that, for the registration, evaluates geometric structures of the patient's eye 15 in the form of a segment of the sclera and in the form of a segment of the cornea.

It should be observed that this registration may be implemented as a matter of principle by virtue of the structures of a portion of the patient's eye 15 from the group of vessel, sclera, segment of the cornea, limbus, conjunctiva vessel being detected and evaluated as geometric structures.

FIG. 11A to FIG. 11D explain the procedure of an ophthalmological operation, in which the surgical tool 30 is used to introduce an incision for the arrangement of the implant into the patient's eye 15.

FIG. 11A shows a plan view of a segment of a patient's eye 15 with an intervention path 97 for introducing an incision into a connection region between the sclera 136 and the cornea 138. The incision allows the implant to be arranged in the patient's eye 15; said implant can be used to remove fluid from the anterior chamber of the patient's eye 15 in order thereby to reduce the intraocular pressure.

FIG. 11B is a partial section of the patient's eye 15 with the intervention path 97. The pose provided for the implant during the ophthalmological operation is identified here by means of a structure 125 drawn using dashed lines. To render this pose possible, it is necessary for a surgeon to align the portion of action 32 of the surgical tool 30 with the intervention path 97 when introducing the incision.

FIG. 11C shows a partial section of the patient's eye 15 with the surgical tool 30. FIG. 11D shows the partial section of the patient's eye 15 with an incision 140 introduced therein and the implant 134. The geometry and pose of the incision 140 in the patient's eye 15 defines the position of the implant 134 in the latter. Following the introduction of the incision 140, the surgeon introduces the implant 134 into the patient's eye 15 using a manipulation tool. FIG. 11E is a partial section of the patient's eye 15 with the implant 134 arranged therein.

For the eye surgery operating system 10′, it is possible to set a mode of operation that serves to facilitate the introduction of an ideal incision into the patient's eye 15 by a surgeon guiding the surgical instrument 30.

To this end, the program memory of the computer unit 36 contains a computer program which, when said mode of operation is set, allows the ascertainment of an actual position of the surgical tool 30 in the object region 14 with the operating site 11 on the patient's eye 15 and of a target location of the portion of action 56 of the surgical tool 54 in the coordinate system 110 referenced to the patient's eye 15 from acquired and predetermined operating system data, in order to display this on the display 78.

FIG. 12 shows a flowchart 101 with program routines of a computer program loaded into a program memory of a computer unit in the eye surgery operating system 10′.

The digital model 84 of the operating site 11 provided in the operating site model program routine 100 and the digital model 86 of the area of effect 76 of the surgical tool 30 provided in the surgical tool program routine 104 are in this case supplied directly to a routine 111 that determines a spatial pose of the model 84 of the operating site relative to the model of the area of effect 86 from referencing measurement data 108 concerning the patient's eye 15 and concerning the surgical tool 30.

The spatial pose of the model 84 of the operating site relative to the model of the area of effect 86 determined in the routine 111 receives a prediction routine 118. From the spatial pose of the model 84 of the operating site relative to the spatial pose of the model of the area of effect 86, the prediction routine 118 calculates a continually adapted model 88 that is valid in a time interval comprising a time window of action in which the surgical tool 30 has an effect on tissue structures in the patient's eye 15.

In the routine 122, the continually adapted model 88 regarding the predicted result of the surgical operation on the patient's eye 15 is assessed and, on account of the assessment, provided as information concerning the continually adapted model 88 in the form of assessment information 142 for positioning the surgical tool 30 by a surgeon, said information being position assessment information.

FIG. 13 shows the display 78 with a first and a second image 144, 146 of the object region 14 in this mode of operation. Concerning the images 144, 146 of the object region 14, the digital model 88 regarding the predicted result of the operation in the form of the pose of the implant 134 relative to the incision into the patient's eye 15 are displayed in each case, corresponding to the pose of the digital model 86 of the area of effect of the surgical tool relative to the digital model of the patient's eye 15.

On the display 78 of the eye surgery operating system 10′, the assessment information is provided as information concerning the model 88 regarding the predicted result of the surgical operation on the patient's eye 15 in the form of a surgical tool positioning signal.

In this case, the surgical tool positioning signal is a graphical marking 135 which displays an unwanted result of the surgery or a negative assessment of a probable result of the surgery. For example using colors, this graphical marking can indicate the degree of an unwanted approach, e.g. yellow, or else conflicting spatial requirements, e.g. red, between the implant and the tissues, e.g. the tissue of the cornea or the tissue of the iris, and the implant to be implanted in the incision potentially created by the surgical tool 30 in the portion of action 76 thereof. The surgical tool 30 is guided by the surgeon by varying the surgical tool position in order to intuitively reduce the negative assessment until an acceptable measure is displayed, e.g. until there is a display that no predicted unwanted approximation is present anymore.

The computer unit 36 in the eye surgery operating system 10′ shown in FIG. 9 in this case contains a signal generator that converts the surgical tool positioning signal into an acoustic signal for a loudspeaker 148. In this way, an offset of the surgical tool 30 from a target position can be indicated to a surgeon during a surgical operation.

It should be observed that the eye surgery operating system 10′ may also contain a signal generator that converts the surgical tool positioning signal into a vibration signal in an alternative to the signal generator that converts the surgical tool positioning signal into an acoustic signal for a loudspeaker 148 or in addition to that. For example, the vibration signal may trigger the vibration of a handle of the surgical tool 30. In this way, it is possible to haptically indicate to a surgeon that there is an offset of the surgical tool 30 from a target position in a surgical operation.

FIG. 14 shows a further eye surgery operating system 10″. To the extent that assemblies and elements of the further eye surgery microscopy system correspond to the assemblies and elements of the eye surgery microscopy systems 10, 10′ described above on the basis of FIG. 1 to FIG. 13, these are identified by the same numbers as reference signs.

The eye surgery operating system 10″ contains a device 150 that is connected to the computer unit 36 and serves to continually measure the intraocular pressure in the patient's eye 15. In the eye surgery operating system 10″, there is a device 152 for irrigating the patient's eye 15. The eye surgery operating system 10″ has a surgical tool 30 that takes the form of a surgical needle, serves for a surgical procedure in the iridocorneal angle and comprises a mirror gonioscope 154 placed against the patient's eye 15.

FIG. 15 shows a curve 156 showing the change in the intraocular pressure IOP in an ophthalmological operation over time t. Typically, the intraocular pressure decreases during ophthalmological operations as a function of time t between irrigations or injections as a consequence of aqueous humor outflow. It should be observed that the curve is an idealized representation since the intraocular pressure may be modulated on the basis of the heartbeat (typical amplitude: 2-4 mmHg). However, this may be taken into account by virtue of the heartbeat being detected using electrodes in operative situations and being used to trigger tonometric measurements with defined relationships to the heartbeat.

FIG. 16A shows the patient's eye 15 in the case of an intraocular pressure IOP above the threshold value S identified in FIG. 14. The procedure in the iridocorneal angle is possible here. FIG. 16B shows the patient's eye 15 in the case of an intraocular pressure IOP below the threshold value S. In this case, the iridocorneal angle of the patient's eye 15 is no longer accessible to the surgical tool 30 that takes the form of a surgical needle, because the patient's eye 15 has sunk into the eye socket in this case.

FIG. 17 is a flowchart 101 with program routines of a computer program loaded into a program memory of a computer unit in the eye surgery operating system 10″.

The digital model 84 of the operating site 11 provided in the operating site model program routine 100 and the digital model 86 of the area of effect 76 of the surgical tool 30 provided in the surgical tool program routine 104 are again supplied to a routine 111 in this case. The routine 111 continually receives referencing measurement data 108 concerning the patient's eye 15 and concerning the surgical tool 30 and the intraocular pressure IOP in the form of intraocular pressure measurement data 158 from the device 150 for continually measuring the intraocular pressure of the patient's eye 15.

From the supplied referencing measurement data 108, the spatial pose of the provided digital model 84 of the operating site 11 is referenced in the routine 111 to the spatial pose of the provided digital model 86 of the area of effect 76 in the surgical operation in a coordinate system 110 of the eye surgery operating system 10, in order thereby to determine, in a prediction routine 118, the continually adapted model 88 which is in regard to the predicted result of the surgical operation on the patient's eye 15 and valid for a time interval comprising the time window of action 124.

The computer program has a routine 122 which in this case assesses the continually adapted model 88 regarding the predicted result of the surgical operation on the patient's eye 15 from the prediction routine 118, giving consideration to the measured intraocular pressure IOP, and provides a display 162 of assessment information as information regarding the continually adapted model 88 on the basis of the assessment, said assessment information indicating to the surgeon whether or not the surgical procedure may be performed on the patient's eye 15.

FIG. 18 shows the display 78 of the further eye surgery operating system 10″ with a digital model of the operating site 11, a digital model of the area of effect of the surgical tool 30 and the display information 162 regarding the performance of the surgical operation for the surgeon. To counteract a reduction in the intraocular pressure IOP of the patient's eye 15, the eye surgery microscopy system 10″ gives a surgeon the option of supplying irrigation fluid to the patient's eye 15 by means of the device 152 for irrigating the patient's eye 15.

It should be observed that a modified embodiment of the eye surgery operating system 10″ may provide for the intraocular pressure or the development thereof over time to be inferred from a change in eye structure poses on the basis of a mechanical eye model in a program routine of the computer program for the computer unit. In this case, it is possible that the mechanical eye model has parameters that are determined from geometry-pressure combinations measured pre-surgery and in surgery, e.g. parameter values from the group of eye size and eye shape for specific different intraocular pressures, in order to use this to infer the intraocular pressure IOP for a specific geometry of the patient's eye 15.

FIG. 19 shows a further eye surgery operating system 10″. To the extent that assemblies and elements of the further eye surgery microscopy system correspond to the assemblies and elements of the eye surgery microscopy systems 10, 10′ and 10″ described above on the basis of FIG. 1 to FIG. 17, these are identified by the same numbers as reference signs.

In the eye surgery operating system 10″, there is a micro-robot 164 for moving the surgical tool 30. The micro-robot 164 has a control unit 166, which is connected to the computer unit 36. By means of the computer unit 36 it is possible to specify for the portion of action 32 of the surgical tool 30 a movement path in a coordinate system 110 referenced to the patient's eye 15, on which the micro-robot 164 moves the portion of action 32 of the surgical tool 30 from an initial position to a start position for a target position in order to automatically perform from there the surgical operation—a trabeculectomy—by means of the surgical tool 30 in the time window of action if a clearance signal FS is present.

FIG. 20 shows a flowchart 101 with program routines of a computer program loaded into a program memory of the computer unit 36 in the eye surgery operating system 10″.

The digital model 84 of the operating site 11 provided in the operating site model program routine 100 and the digital model 86 of the area of effect 76 of the surgical tool 30 provided in the surgical tool program routine 104 are once again supplied to a routine 111 that determines a spatial pose of the model 84 of the operating site relative to the model of the area of effect 86 from referencing measurement data 108 concerning the patient's eye 15 and concerning the surgical tool 30. The calculated spatial pose of the model 84 of the operating site relative to the model of the area of effect 86 receives a prediction routine 118, in which is determined from the spatial pose of the provided digital model 84 of the operating site 11 relative to the spatial pose of the provided digital model 86 of the area of effect 76 a continually adapted model 88 in regard to the predicted result of the surgical operation on the patient's eye 15 and valid for a time interval comprising the time window of action.

The routine 122 of the computer program assesses the predicted result of the surgical operation and provides the clearance signal FS as a guidance signal to the control unit 166 in the presence of assessment information corresponding to positive assessment of the predicted result of the surgical operation on account of an assessment criterion and said guidance signal controls the micro-robot 164 in such a way that the user automatically performs the trabeculectomy as a surgical procedure on the patient's eye 15 predetermined therefor.

FIG. 21 is a representation of a segment of a patient's eye 15 following a trabeculectomy with a relief channel 170 that is automatically prepared by means of the eye surgery operating system 10′ and in which eye fluid can flow out of the interior of the patient's eye 15 in the direction of the arrows 172 into a relief volume 174 (“bleb”).

In conclusion, the following preferred features should, in particular, be retained: An eye surgery operating system 10 for performing a surgical operation in an operating site 11 on a patient's eye 15 contains a surgical tool 30 with which it is possible to have an effect on tissue structures of the patient's eye 15 that are arranged in an area of effect 76, which is a spatially extensive region of possible effects due to the surgical tool 30 within a time window of action 124. The eye surgery operating system 10, 10′, 10″, 10″ has a computer unit 36 which contains a program memory with a computer program that comprises an operating site model program routine 84 for providing a model of the operating site 11. The computer unit 36 is embodied to continually acquire referencing measurement data 108 concerning the patient's eye 15 and concerning the surgical tool 30. The computer program comprises a prediction routine 118 developed to use the spatial pose of the model 84 of the operating site 84 relative to the spatial pose of the model of the area of effect 86 to determine a continually adapted model that is in regard to the predicted result of the surgical operation on the patient's eye 88 and valid for a time interval 126 comprising the time window of action 124. The computer program contains a routine 122 for continually providing assessment information relating to the guidance of the surgical tool 30 in the surgical operation, said routine considering the model of the area of effect 86 and the model of the operating site 84 and the provided, continually acquired referencing measurement data 108 and the model regarding the predicted result of the surgical operation on the patient's eye 88.

### LIST OF REFERENCE SIGNS

10, 10′, 10″, 10′″ Eye surgery operating system
11 Operating site
12 Surgical microscope
14 Object region
15 Patient's eye
16 Main microscope objective system
18 Illumination device
20 Magnification system
22 First partial stereoscopic observation beam path
22 Left stereoscopic observation channel
24 Second partial stereoscopic observation beam path
24 Right stereoscopic observation channel
25 Optical axis
26 Binocular tube
28 Operating unit
30 Surgical tool
32 Portion of action
36 Computer unit
38 Apparatus for providing stereoscopic images
40 OCT apparatus
42 Scheimpflug camera
44 First image capturing device
46 Objective lens system
48 Image sensor
50 Second image capturing device
52 Image calculating stage
54 Object region volume
56 OCT scanning beam
58, 60 Scanning mirror
62, 64 Beam splitter
66 Control device
68 Scheimpflug camera plane
70 Arrow
72, 74 Marking
76 Area of effect
77, 77′ Device for reflecting data
78 Display
79 User interface
80, 82 View
84 Model of the operating site
84′ Representation of the model of the operating site
86 Model of the area of effect
86′, 86″ Representation of the model of the area of effect
88 Model regarding the predicted result of the surgery
88′, 88″ Representation of the model for the predicted result of the surgery
89 Displacement information
90 Movement path
91 Model for an optimal result of the surgery
92 Tip
94 Initial pose
96 Intervention pose
97 Intervention path
98 Acceptance region
99 Arrow
100 Operating site model program routine
101 Flowchart
102 Operating site pose routine
104 Surgical tool program routine
106 Surgical tool pose routine
108 Referencing measurement data
110 Coordinate system
111 Routine for relative pose of the models
112 Referencing routine
114 Target state pose routine
116 Displacement information routine
118 Prediction routine
122 Routine for assessment information
120, 123 Display routine
124 Time window of action
125 Structure
126 Time interval
128 Time
130 Time axis
132 Referencing time
134 Implant
135 Marking
136 Sclera
138 Cornea
140 Incision
142 Pose assessment information
144, 146 Image
148 Loudspeaker
150 Device for measuring the intraocular pressure
152 Device for irrigation
154 Mirror gonioscope
156 Curve
158 Intraocular pressure measurement data
162 Display
164 Micro-robot
166 Control unit
170 Relief channel
172 Arrows
174 Relief volume
lz Length of the time interval comprising the time window of action
lw Length of the time window of action

1. An eye surgery operating system for performing a surgical operation in an operating site on a patient's eye, the eye surgery operating system comprising:
a surgical tool with which it is possible to have an effect on tissue structures arranged in an area of effect on the patient's eye, wherein the area of effect is a spatially extensive region of possible effects due to the surgical tool within a time window of action,
a computer unit including a computer-readable memory storing a computer program, the computer unit configured to execute the computer program to:
provide a model of the operating site;
continually acquire referencing measurement data concerning the patient's eye and concerning the surgical tool;
provide a model of the area of effect;
determine a spatial pose of the model of the operating site relative to the model of the area of effect from the continually acquired referencing measurement data;
determine a continually adapted model that is in regard to a predicted result of the surgical operation on the patient's eye and valid for a time interval comprising the time window of action based on the spatial pose of the model of the operating site relative to a spatial pose of the model of the area of effect; and
continually provide assessment information relating to guidance of the surgical tool in the surgical operation in consideration of the model of the area of effect, the model of the operating site, the continually acquired referencing measurement data, and the model regarding the predicted result of the surgical operation on the patient's eye.

2. The eye surgery operating system as claimed in claim 1, wherein the time window of action has a length lw, to which the following applies: 16 picoseconds (ps)≤lw≤0.4 second (s).

3. The eye surgery operating system as claimed in claim 1, wherein the following applies to a length lz of the time interval comprising the time window of action and a length lw of the time window of action: lz>lw+0.1 s

4. The eye surgery operating system as claimed in claim 3, wherein the following applies to the length lz of the time interval comprising the time window of action:
lz>L,
where L is a latency time for a provision of the model regarding the predicted result of the surgical operation on the patient's eye.

5. The eye surgery operating system as claimed in claim 1, wherein the computer unit is further configured to execute the computer program to continually adapt the model of the operating site based on the continually acquired referencing measurement data.

6. The eye surgery operating system as claimed in claim 1, wherein the model regarding the predicted result of the surgical operation on the patient's eye is a model for a pose of an implant in the patient's eye.

7. The eye surgery operating system as claimed in claim 6, wherein the assessment information relating to the guidance of the surgical tool contains assessment information regarding an expected result of a surgery or is assessment information regarding an expected result of the surgery that is ascertained from a comparison of the model for the predicted result of the surgical operation with a reference.

8. The eye surgery operating system as claimed in claim 7, further comprising a device for indicating the assessment information regarding an expected result of the surgery as an acoustic and/or an optical and/or a haptic indication signal.

9. The eye surgery operating system as claimed in claim 7, wherein the reference is a model for an optimal result of the surgery, created for the patient's eye.

10. The eye surgery operating system as claimed in claim 9, wherein the model for the optimal result of the surgery created for the patient's eye is based on patient data acquired pre-surgery.

11. The eye surgery operating system as claimed in claim 1, wherein the model of the operating site is a point cloud that describes the operating site and/or a surface shape of a cornea of the patient's eye and/or a CAD model and/or a height profile of a segment of the patient's eye and/or a distance profile of the patient's eye and/or a depth profile of the patient's eye and/or a three-dimensional surface representation of a segment of the patient's eye and/or a two-dimensional surface representation of a segment of the patient's eye.

12. The eye surgery operating system as claimed in claim 1, wherein the model of the area of effect is a point cloud that describes a zone in which the surgical tool can have an effect on body tissue in the patient's eye and/or on media arranged in the patient's eye.

13. The eye surgery operating system as claimed in claim 1, wherein the computer unit is further configured to execute the computer program to continually provide displacement information relating to the guidance of the surgical tool in the surgical operation and is ascertained from the model of the area of effect and the model of the operating site and the provided, continually acquired referencing measurement data.

14. The eye surgery operating system as claimed in claim 13, wherein the displacement information relating to the guidance of the surgical tool is information from the group of spatial pose of the surgical tool and direction for displacement of the surgical tool in a coordinate system of the eye surgery operating system or relative to the model of the operating site.

15. The eye surgery operating system as claimed in claim 14, further comprising a device for indicating the displacement information as an acoustic and/or an optical and/or a haptic indication signal.

16. The eye surgery operating system as claimed in claim 1, further comprising a device that is coupled to the computer unit and measures intraocular pressure in the patient's eye, wherein the assessment information is based, at least in part, on the measured intraocular pressure.

17. The eye surgery operating system as claimed in claim 13, further comprising a device that is coupled to the computer unit and measures intraocular pressure in the patient's eye wherein the displacement information is based, at least in part, on the measured intraocular pressure.

18. The eye surgery operating system as claimed in claim 17, further comprising a micro-robot having a control unit that receives the displacement information from the computer unit.

19. The eye surgery operating system as claimed in claim 15, further comprising a micro-robot having a control unit that receives the displacement information from the computer unit.

20. The eye surgery operating system as claimed in claim 1, wherein the computer unit is further configured to execute the computer program to assesses characteristic surgical tool features of opacity, casted shadow, edge or surface shape, light signals based on image recognition and/or markings on the surgical tool.

21. The eye surgery operating system as claimed in claim 20, wherein the computer unit is further configured to execute the computer program to consider a refraction of light at interfaces in the patient's eye and/or index gradients in the patient's eye.

22. The eye surgery operating system as claimed in claim 1, further comprising a magnetic tracking system, wherein the referencing measurement data contain location data concerning the surgical tool that were acquired by the magnetic tracking system.

23. The eye surgery operating system as claimed in claim 1, wherein the surgical tool is a surgical tool from the group of lancet, laser, needle, stabilized needle, drill, injection needle, endoscope having a laser for tissue ablation or tissue coagulation, implant injector, goniotomy cutter, trabecular meshwork trephine, plasma cutter.

24. A non-transitory computer-readable storage medium storing a computer program for providing assessment information relating to guidance of a surgical tool in a surgical operation on a patient's eye, the computer program, when executed by a computer unit, causes the computer unit to:
provide a model of an operating site;
provide a model of an area of effect of a surgical tool, which describes a spatially extensive region of possible effects due to the surgical tool within a time window of action,
determine a spatial pose of the model of the operating site relative to the model of the area of effect from continually acquired referencing measurement data,
determine a continually adapted model that is in regard to a predicted result of the surgical operation on the patient's eye and valid for a time interval comprising the time window of action based on the spatial pose of the model of the operating site relative to the spatial pose of the model of the area of effect; and
continually provide the assessment information relating to the guidance of the surgical tool in consideration of the model of the area of effect, the model of the operating site, the continually acquired referencing measurement data, and the model regarding the predicted result of the surgical operation on the patient's eye.

25. A computer-implemented method for providing assessment information for the assessment of a surgical tool in a surgical operation on a patient's eye, the method comprising:
providing a model of an operating site;
providing a model of an area of effect of the surgical tool, which describes a spatially extensive region of possible effects due to the surgical tool within a time window of action;
determining the spatial pose of the model of the operating site relative to the model of the area of effect from continually acquired referencing measurement data;
determining a continually adapted model that is in regard to the predicted result of the surgical operation on the patient's eye and valid for a time interval comprising the time window of action, from the spatial pose of the model of the operating site relative to the spatial pose of the model of the area of effect; and
providing the assessment information relating to the guidance of the surgical tool considering the model of the area of effect, the model of the operating site, the continually acquired referencing measurement data, and the model regarding the predicted result of the surgical operation on the patient's eye.
